(19) United States
(12) Patent Application Publication
(10) Pub. No.: US 2012/0130406 A1
(43) Pub. Date: May 24, 2012

(54) COATED THREAD WITH ANCHORING STRUCTURES FOR ANCHORING IN BIOLOGICAL TISSUES

(75) Inventors: Erich Odermatt, Schaffhausen (CH); Ingo Berndt, München (DE); Silke König, Rottweil (DE); Sven Oberhoffner, Weinstadt-Endersbach (DE); Erhard Müller, Stuttgart (DE)

(73) Assignees: ITV DENKENDORF PRODUKTSERVICE GMBH, Denkendorf (DE); AESCULAP AG, Tuttlingen/Donau (DE)

(21) Appl. No.: 13/318,893

(22) PCT Filed: May 10, 2010
(86) PCT No.: PCT/EP10/02847

§ 371 Date: Feb. 9, 2012

(30) Foreign Application Priority Data
May 8, 2009 (DE) 10 2009 020 901.8

(51) Int. Cl. A61B 17/03; B05D 5/00; A61B 17/04

(52) U.S. Cl. 606/151; 606/228; 427/2.31

(57) ABSTRACT

Thread for use as a knotless or self-fixing surgical suture material including a main body, and anchoring structures formed on a surface of the main body of the thread and anchored in human or animal tissues, wherein at least some of the anchoring structures are at least partially covered by a coating of the main body of the thread, which coating is removable by liquids.

### RELATED APPLICATIONS

This is a §371 of International Application No. PCT/EP2010/002847, with an international filing date of May 10, 2010 (WO 2010/127874 A1, published Nov. 11, 2010), which is based on German Patent Application No. 10 2009 020 901.8, filed May 8, 2009, the subject matter of which is incorporated by reference.

### TECHNICAL FIELD

This disclosure relates to a thread suitable, in particular, for use as a knotless or self-fixing surgical suture material, to a method for producing the thread, and to surgical uses of the thread.

### BACKGROUND

Thread-like suture materials are used as standard in surgery for closure of wounds. They are usually knotted to obtain a secure fixing in the tissue. Care has to be taken to ensure that the wounds to be closed are sutured with an optimal force at the wound margins. If the wound margins are sutured too loosely and too irregularly, for example, there is in principle a risk of increased scar formation or dehiscence. In contrast, if the wound margins are sutured too strongly, there is a danger of the circulation of blood in the wound margins being restricted, which can result in necrotic changes in the surrounding tissue area.

In addition to the risk of possible complications, in particular, further surgical interventions, there is therefore always a degree of risk of the wound repair, based on knotting of suture materials, leading to impaired healing and to unsatisfactory cosmetic results in the patients concerned. Another consideration is that several knots often have to overlap to achieve a secure knot hold. This entails introducing a large amount of material into the area of the wound that is to be treated and can lead to increased foreign-body reactions, particularly in the case of resorbable suture materials.

Suture materials which, in contrast to conventional threads, do not have to be knotted have long been known under the term “barbed sutures.” Such knotless or self-fixing suture materials are usually composed of a monofilament thread which, along its longitudinal axis, has structures called “barbs.” Corresponding suture materials are described, for example, in U.S. Pat. No. 3,123,077 A, EP 1 559 266 B1, EP 1 560 683 B1 and EP 1 556 946 B 1. The barbs are usually formed on a thread such that the thread can be pulled through a tissue along the direction of the barbs. When a pull force is exerted in the opposite direction, the barbs can stand upright and anchor themselves, and therefore also the thread, in the surrounding tissue area. This ensures that the thread cannot be pulled back through the incision channel.

Although the thread, as mentioned in the preceding paragraph, is generally pulled through a biological tissue along the direction of its barbs, tissue trauma caused by the barbs can never be entirely ruled out. To overcome this problem, the use of a tubular insertion device is proposed in U.S. Pat. No. 6,241,747 B1, U.S. Pat. No. 5,342,376 and DE 10 2005 004 318 A1. The insertion device in the first instance avoids direct contact of the barbs with the body tissue. It is only after the suture material has been correctly positioned that the insertion device is removed, exposing the barbs, and the barbs are able to anchor themselves in the surrounding tissue area. The use of insertion devices, however, makes surgical techniques employing them complicated and susceptible to error.

It could therefore be helpful to provide a knotless or self-fixing suture material that can be pulled through tissue with the least possible trauma, and without the need to use additional aids.

### SUMMARY

We provide thread for use as a knotless or self-fixing surgical suture material including a main body, and anchoring structures formed on a surface of the main body of the thread and that are used for anchoring in human or animal tissues, wherein at least some of the anchoring structures are at least partially covered by a coating of the main body of the thread, which coating is removable by liquids.

We also provide a surgical implant including at least one thread.

We further provide a surgical kit including at least one surgical insertion instrument and at least one thread.

We further yet provide a method for producing a thread, including applying a coating that is removable by liquids to a surface of a main body of the thread, on the surface of which main body anchoring structures are formed to anchor in human or animal tissues.

We also further provide a mesh including a plurality of threads.

### BRIEF DESCRIPTION OF THE DRAWINGS

FIG. 1 shows an example of our thread.

FIG. 2 shows another example of our thread.

### DETAILED DESCRIPTION

Our thread is a thread comprising a main body, and anchoring structures that are used for anchoring in biological tissues, in particular, human and/or animal tissues, and that are formed on the surface of the main body of the thread. The main body of the thread is generally designed as an elongate body. At least some of the anchoring structures are covered at least partially, preferably completely, by a coating of the main body of the thread, which coating can be removed by means of liquids, in particular, body fluids. Preferably, all the anchoring structures are covered by the coating of the main body of the thread. The thread is suitable, in particular, as a knotless or self-fixing surgical suture material.

Biocompatible liquids, or preferably body fluids themselves, are preferable as the liquids for removing the coating. Examples of suitable biocompatible liquids are chosen from the group consisting of water, salt solutions, electrolyte solutions, buffer solutions and sugar solutions. Body fluids are to be understood, in particular, herein as tissue fluid, blood, lymph, wound fluid or exudate.

The biological tissues can be, for example, skin, fat, fascias, bones, muscles, organs, nerves, blood vessels, connective tissues, tendons or ligaments.

Preferably, at least some of the anchoring structures are embedded, preferably completely embedded, in the coating.

The anchoring structures are generally fixed by the coating in a certain position on the surface of the main body of the thread. Preferably, at least some of the anchoring structures are fixed by the coating in a position protruding from the surface of the main body of the thread. Particularly preferably, all the anchoring structures are fixed by the coating in a position protruding from the surface of the main body of the thread. In particular, the anchoring structures may be pressed onto the surface of the main body of the thread.

Alternatively, at least some of the anchoring structures are fixed by the coating in a position bearing on the surface of the main body of the thread. In other words, at least some of the anchoring structures do not protrude from the surface of the main body of the thread. It is particularly preferable if all the anchoring structures are fixed by the coating in a position bearing on the surface of the main body of the thread.

The coating can preferably dissolve in liquids or upon contact with liquids or is soluble in such liquids. The coating can preferably dissolve in or upon contact with body fluids or is soluble in body fluids. This advantageously avoids the need for measures for removing the coating, which measures would otherwise have to be taken by a user, for example, by a surgeon. It is particularly advantageous if the coating dissolves in or upon contact with body fluids only after a certain period of time. In this way, the user of the thread is given sufficient time to carry out a repositioning of the thread if this proves necessary. For example, the coating can be designed such that it dissolves in or upon contact with body fluids only after a few minutes. In other words, it is particularly preferable if the coating is made from a material that dissolves in or upon contact with body fluids but after some delay.

However, it is also possible in principle for the coating to dissolve in or upon contact with biocompatible liquids or to be soluble in such liquids. In this way, after correct positioning of the thread, the coating can be removed, for example, by a simple flushing procedure. It is also possible for the examples described in the preceding paragraph to be combined with a flushing procedure, if appropriate, to accelerate removal of the coating.

After removing the coating, the anchoring structures preferably protrude from the main body of the thread.

The coating itself is preferably formed in the manner of a sheath on the surface of the main body of the thread. The coating preferably completely surrounds the main body of the thread, including the anchoring structures. The coating is generally formed with a uniform layer thickness on the surface of the main body of the thread. The coating preferably has a radius component of 5 to 100%, in particular, 10 to 50%, relative to a radius of the main body of the thread (without anchoring structures protruding therefrom). In principle, the thread can have a coating that amounts to 3 to 70% by weight relative to the total weight of the thread. Small amounts of coating are preferred, since the removal of the coating can be accelerated in this way. With the coating, the thread preferably has a friction-reducing outer surface, preferably a substantially flat outer surface.

Moreover, the coating can be configured as a film, sleeve, sheath, encasing, membrane, sponge, foam or gel, in particular hydrogel, on the surface of the main body of the thread.

Preferably, the coating is configured as a tube or hose. More preferably, the tube or hose comprises anchoring structures. In principle, the anchoring structures may protrude into the interior and/or exterior of the tube or hose. Preferably, the anchoring structures protrude into the exterior of the tube or hose. Further, the anchoring structures may be configured as cuts into the surface of the main body, wherein the cuts preferably do not break through the wall of the tube or hose. As an alternative or in combination, the anchoring structures may be configured as breakthroughs, i.e., the anchoring structures are formed completely breaking through the wall of the tube or hose. A coating configured as a tube or hose having anchoring structures is especially advantageous since it imparts the thread self-anchoring properties even immediately upon implantation of the thread into the body of a patient. A tubular or hose-like coating may be applied on the thread by shrinkage and/or drawing. In general, a tubular or hose-like coating having anchoring structures as described herein may be applied in particular shrunk and/or drawn, on a round stock, fiber, monofilament, pseudomonofilament, multifilament, thread, yarn or the like to equip these structures with self-anchoring properties. To accelerate the dissolution of the coating by liquids, in particular, body liquids and/or biocompatible liquids, the coating may have a small wall thickness.

In another example, the coating is porous, in particular, with open pores. The coating preferably has a porosity of 30 to 90%, in particular 60 to 80%, relative to the total volume of the coating. The higher the porosity of the coating, the less coating material has to be removed to expose the anchoring structures on the surface of the main body of the thread.

In yet another example, the main body of the thread and the anchoring structures are formed integrally. The anchoring structures are preferably formed as cuts in the surface of the main body of the thread. As has already been mentioned, the anchoring structures can protrude from the surface of the main body of the thread. The cuts can be mechanical cuts, physicochemical cuts, in particular, laser-generated cuts, or thermal cuts. To form mechanical cuts, cutting blades can be used, for example. Suitable cutting blades are often part of a cutting device, which additionally comprises a cutting board (cutting abutment) and also holding or fixing elements, for example, a vice, chucks, holding or clamping jaws, for the thread that is to be cut. Thermal cuts can be generated, for example, by a heated cutting blade or a heated cutting wire, in particular an electrically heated cutting wire. To form laser-generated cuts, it is possible in principle to use gas lasers, for example, CO₂ lasers, and also solid-state lasers, for example, Nd:YAG lasers. Corresponding measures for generating anchoring structures on thread surfaces are sufficiently familiar such that further details are not needed here.

Alternatively, the anchoring structures are formed integrally on the main body of the thread such that they protrude permanently from the surface of the main body of the thread, i.e., cannot be converted to a position flush with the surface of the main body of the thread. Corresponding anchoring structures can be formed on the surface of the main body of the thread by injection molding, for example. In particular, the thread can be produced by injection molding.

The anchoring structures may be hook-shaped, in particular, barb-shaped, escutcheon-shaped, shield-shaped, scale-shaped, wedge-shaped, thorn-shaped, arrow-shaped, V-shaped and/or W-shaped, on the surface of the main body of the thread. The anchoring structures are particularly preferably barb-shaped or designed in the manner of barbs or designed as barbs on the surface of the main body of the thread. At their ends protruding away from the surface of the main body of the thread, the anchoring structures can also be sharp or pointed to facilitate penetration into tissues.

The anchoring structures can in principle be formed in different arrangements on the surface of the main body of the thread. For example, the anchoring structures can have a linear arrangement, a row by row arrangement, an offset arrangement, a zigzag arrangement, a spiral-shaped arrangement, a helical-shaped arrangement, a random arrangement, or combinations of these, in the longitudinal and/or transverse direction, preferably in the longitudinal direction, of the thread. The abovementioned arrangements are generally visible to the user only after removal of the coating. An arrangement is particularly preferred in which the anchoring structures are distributed across the entire surface of the main body of the thread, since in this case, after removal of the coating, the thread can anchor itself particularly firmly in a surrounding tissue area.

The thread may have at least one set, in particular two, three or more sets, of anchoring structures on the surface of the main body of the thread. A set of anchoring structures is to be understood as an arrangement of anchoring structures on the surface of the main body of the thread that corresponds in respect of the configuration of the anchoring structures, in particular in respect of the height of the anchoring structures, the length of the anchoring structures, the cutting depth of the anchoring structures, the angle at which the anchoring structures protrude from the surface of the main body of the thread, the orientation of the anchoring structures and/or the shape of the anchoring structures. The number of sets and the configuration of their anchoring structures are also usually recognizable only after removal of the coating.

The anchoring structures can be formed unidirectionally on the surface of the main body of the thread. Preferably, the anchoring structures are formed bidirectionally on the surface of the main body of the thread. A bidirectional arrangement of anchoring structures is to be understood here as an arrangement in which the anchoring structures are formed in two different directions (bidirectionally) on the surface of the main body of the thread. Preferably, in the longitudinal direction of the thread, the anchoring structures for a first thread portion are formed in the direction of another, second thread portion, and the anchoring structures for the other, second thread portion are formed in the direction of the first thread portion. For example, seen in the longitudinal direction of the thread, the anchoring structures for a first thread portion can be oriented in the direction of the center of the thread and, for another, second thread portion, are likewise oriented in the direction of the center of the thread. The length of the thread portions can correspond to approximately half the thread length such that the thread center forms a kind of center of symmetry.

Particularly advantageously, the surface of the main body of the thread has at least two bidirectional arrangements of anchoring structures. In this case, it is preferable if, in relation to a first bidirectional arrangement of anchoring structures, a second bidirectional arrangement of anchoring structures is formed on the surface of the main body of the thread at approximately 180 degrees in the circumferential direction and preferably offset in relation to the first bidirectional arrangement. It is also possible for the thread to have a total of three bidirectional arrangements of anchoring structures. In this case, it is preferable if, in relation to a first bidirectional arrangement of anchoring structures, a second bidirectional arrangement of anchoring structures is formed on the surface of the main body of the thread at approximately 120 degrees in the circumferential direction and preferably offset in relation to the first bidirectional arrangement, which second bidirectional arrangement of anchoring structures is in turn formed at approximately 120 degrees in the circumferential direction of the thread and preferably offset in relation to a third bidirectional arrangement of anchoring structures such that the third bidirectional arrangement of anchoring structures is likewise formed at approximately 120 degrees in the circumferential direction of the thread and preferably offset in relation to the first bidirectional arrangement of anchoring structures.

The anchoring structures may have a periodically changing, preferably alternating, orientation on the surface of the main body of the thread. For example, seen in the longitudinal direction of the thread, the anchoring structures for a first thread portion can be oriented in the direction of a second thread portion and, for the second thread portion, in the direction of the first thread portion, and for a third thread portion adjoining the second thread portion can be formed in the direction of a fourth thread portion adjoining the third thread portion and, for the fourth thread portion, can be formed in the direction of the third thread portion and the like.

The coating of the thread can soften or melt in or upon contact with body fluids. It is particularly advantageous if the coating is converted to a low-viscosity melt. This has the advantage that the coating, after it has melted, can diffuse away from the surface of the main body of the thread relatively quickly and/or can be flushed away as a result of which the anchoring structures on the surface of the main body of the thread are exposed.

The coating generally comprises a biocompatible material, in particular a biocompatible oligomer and/or polymer. The coating can, in particular, be composed mainly of the coating material. The coating material can be a synthetic polymer and/or naturally occurring polymer or biopolymer. Moreover, the polymer can be a copolymer. A “copolymer” is to be understood as a polymer composed of two or more monomer units. Accordingly, copolymers can also be terpolymers or tetrapolymers. The polymer can, in particular, be a random copolymer or a block copolymer. The polymer can also be a block terpolymer. Polymers with a glass transition temperature of 30 to 37° C. are preferred.

The coating material is preferably chosen from the group consisting of proteins, polysaccharides, polyhydroxyalkanoates, oligohydroxyalkanoates, salts thereof, derivatives thereof, and combinations thereof. The polysaccharides can be chosen, for example, from the group comprising alkyl celluloses, hydroxyalkyl celluloses, carboxyalkyl celluloses, glycosaminoglycans, salts thereof, derivatives thereof, and combinations thereof. Preferably, the coating material is chosen from the group consisting of polyvinyl alcohol (PVA), polyvinylpyrrolidone, collagen, gelatin, elastin, reticulin, albumin, dextran, amylose, amylose pectin, starch, chitosan, methyl cellulose, carboxymethyl cellulose, hydroxymethyl cellulose, hydroxyethyl cellulose, hydroxyethyl methyl cellulose, hydroxypropyl cellulose, hydroxypropyl methyl cellulose, hydroxybutyl cellulose, hyaluronic acid, heparin, heparin sulphate chondroitin-4 sulphate, chondroitin-6 sulphate, dermatan sulphate, keratan sulphate, salts thereof, derivatives thereof, and combinations thereof.

The abovementioned polyhydroxyalkanoates may be chosen from the group consisting of polylactide, polyglycolide, polytrimethylene carbonate, polycaprolactone, poly-p-dioxanone, poly-3-hydroxybutyrate, poly-4-hydroxybutyrate, copolymers thereof, and mixtures thereof. The oligohydroxyalkanoates cited in the preceding paragraph can include lactide, glycolide, trimethylene carbonate, caprolactone and/or p-dioxanone.

The main body of the thread and the anchoring structures can in principle be formed from all the materials suitable for this purpose, preferably polymers. The main body of the thread and the anchoring structures can comprise different materials, in particular different polymers, or can be formed from different materials, in particular different polymers. The main body of the thread and the anchoring structures preferably comprise the same materials, in particular the same polymers, or are formed from the same materials, in particular the same polymers. The polymers can be resorbable, partially resorbable or non-resorbable polymers. The polymers can be present as homopolymers, copolymers, terpolymers or tetrapolymers. The polymers can also be block polymers, in particular block copolymers or block terpolymers. The use of random or alternating copolymers or terpolymers is possible.

If the main body of the thread and/or the anchoring structures are formed from resorbable polymers, the polymers are preferably chosen from the group including polylactide, polyglycolide, poly-ε-caprolactone, poly-para-dioxanone, polytrimethylene carbonate, polyhydroxybutyrate, copolymers thereof, terpolymers thereof and mixtures thereof. A suitable polyhydroxybutyrate is poly-3-hydroxybutyrate and/or poly-4-hydroxybutyrate. Resorbable copolymers or terpolymers are particularly preferred, in particular, resorbable block copolymers or block terpolymers which comprise a monomer from the group including lactide, glycolide, trimethylene carbonate, para-dioxanone, ε-caprolactone, 3-hydroxybutyrate, 4-hydroxybutyrate, and combinations thereof. For example, the main body of the thread and/or the anchoring structures can be made from a triblock terpolymer, comprising glycolide, trimethylene carbonate and ε-caprolactone. A triblock terpolymer of this kind is commercially available under the name Monosyn®.

The non-resorbable materials used for the main body of the thread and/or the anchoring structures can be polymers from the group including polyolefins, polyesters, polyamides, polyurethanes, copolymers thereof, terpolymers thereof, and mixtures thereof. Polypropylene, polyethylene terephthalate, polypropylene terephthalate, polybutylene terephthalate, polytetrafluoroethylene, polyvinylidendifluoride, polytetra-fluoropropylene, polyhexafluoropropylene, linear and preferably aliphatic polyurethanes and/or nylon are mentioned by way of example.

The thread can contain active substances, in particular antimicrobial, disinfecting, anti-inflammatory, analgesic, growth-promoting and/or deodorizing active substances. The active substances can be contained in the main body of the thread, in the anchoring structures and/or in the coating. However, the active substances are generally contained in the main body of the thread and/or in the anchoring structures since in this case they are able to act for longer in the body of a human or animal patient.

The thread, in particular the main body, may be a mass or solid thread, in particular a mass or solid main body. This preferably means that the thread, in particular the main body, has no lumen.

The thread may be designed as a hollow thread, in particular as a tubular thread, preferably as a tube or hose. Preferably, the hollow thread comprises a closed wall, wherein the ends of the thread are preferably open. Such a hollow thread may be produced, by way of example, by extrusion. The anchoring structures may be designed as cuts into the wall of the hollow thread, wherein the cuts preferably do not break through the wall of the hollow thread. As an alternative or in combination, the anchoring structures may be designed as breakthroughs, i.e., the anchoring structures may be formed completely breaking through the wall of the hollow thread. Hollow threads may be used as self-anchoring infusion tubes, delivery tubes, catheters, distribution systems for medical agents, in particular liquid medical agents, drug-release-systems and/or drainage systems, in particular drainage tubes.

More specifically, the main body may be present as a hollow main body, in particular a tubular main body, preferably as a tube or hose. With respect to further details and advantages, reference is made in its entirety to the previous description.

The thread, in particular the main body of the thread, generally has a circular cross section. However, other cross-sectional shapes are also conceivable in principle. For example, the thread, in particular the main body of the thread, can have an oval, triangular, triple-lobed, square, trapezoidal, rhomboid, pentagonal, hexagonal, star-shaped or cross-shaped cross section.

The thread can in principle be a monofilament, a pseudomonofilament or multiilament. If the thread is a multifilament, in particular multifilament yarn, the thread can additionally be present in a braided form. However, it is particularly preferble if the thread is a monofilament.

The thread is preferably a surgical suture material, in particular a knotless or self-fixing surgical suture material.

The thread may be armed at least at one end, in particular at both ends, with a surgical insertion instrument, generally a surgical needle. It is particularly advantageous if the surgical insertion instrument for arming the thread has a hole into which the thread can be introduced. After the thread has been introduced into the hole, the surgical insertion instrument can then be squeezed together or crimped in the area of the bore.

The thread is generally present in a stretched form. This is especially the case when the thread is designed as surgical suture material. In principle, however, it is also possible for the thread to be present in an unstretched form.

The thread may be in a sterilized and ready-to-use state and, in particular, packaged.

We further provide a surgical implant that preferably comprises at least one thread. The implant is preferably formed as a textile implant. Examples of possible implants are chosen from the group including hernia meshes, prolapse meshes, urinary incontinence bands and wound dressings. However, the surgical implant is particularly preferably formed as surgical suture material comprising at least one thread.

We also provide a surgical kit. The kit comprises at least one surgical insertion instrument, in particular a surgical needle or a surgical cannula, and at least one thread.

We further provide a method for producing a thread. To produce the thread, a coating that can be removed by liquids, in particular body fluids, is applied to the surface of a main body of the thread, on the surface of which main body anchoring structures are formed for anchoring in biological tissues, in particular human and/or animal tissues.

Preferably, the coating is applied to the surface of the main body of the thread by sheathing extrusion, as a result of which at least some of the anchoring structures, preferably all of them, are covered, in particular embedded. The technique of sheathing extrusion of threads is sufficiently familiar and no further details are therefore given at this point.

Alternatively, the coating is applied to the surface of the main body of the thread by the following steps:
a) immersing the main body of the thread in a solution or suspension that contains a coating material;
b) removing the main body of the thread from the solution or suspension; and
c) drying the thread body in order to form the thread.

Water, methanol, ethanol, propanol, isopropanol and/or acetone are suitable in principle as solvents for preparing a solution or suspension containing the coating material. The coating material can be present in the solution or suspension at a concentration of 5 to 70% by weight, in particular 10 to 50% by weight, relative to the total weight of the solution or suspension. The coating material precipitates during immersion of the main body of the thread into the solution or suspension, as a result of which a deposit forms on the main body of the thread to be coated, which deposit preferably completely covers the main body of the thread. The precipitation of the coating material can be brought about, for example, by a change of the concentration, temperature and/or pH value of the solution or suspension. If appropriate, step a) of the method can also be repeated several times to achieve, for example, a complete coating of the main body of the thread and/or a greater layer thickness.

To apply the coating, the main body of one form of our thread can be pulled through a tubular auxiliary instrument, the diameter of which corresponds to the diameter of the main body of the thread without anchoring structures and, as the main body of the thread emerges from the auxiliary instrument, the coating is applied directly to the surface of the main body of the thread. If the main body of the thread is pulled through an auxiliary instrument of this kind, the anchoring structures (protruding from the main body of the thread) are pressed onto the surface of the main body of the thread. The anchoring structures can be fixed or held in this position by the applied coating. It is also possible in principle for the coating material to be injected through nozzles into the auxiliary instrument.

The main body of the thread, before being immersed in a solution or suspension containing a suitable coating material, may be introduced into a tubular auxiliary instrument which is provided with openings and of which the diameter corresponds to the diameter of the main body of the thread without anchoring structures. The auxiliary instrument (including the inserted main body of the thread) may then be immersed in the solution or suspension.

The thread can be dried in principle at room temperature, in a heating oven, by a hot current of air, infrared radiation or other known techniques.

Finally, we provide for the use of the thread to produce a surgical implant, in particular a surgical suture material, preferably a knotless or self-fixing surgical suture material. The surgical implant, preferably surgical suture material, is suitable especially for fixing or tightening biological tissue, in particular human or animal tissue. A preferred area of use of the thread is plastic surgery. There, the thread is preferably used for tightening the skin or for lifting, for example, for eyebrow lifts. Other areas of use are for correction of the cheek and/or chin lines. In addition, the thread is also suitable for other surgical indications, in particular for indications in which the use of conventional suture materials is difficult on account of steric hindrance. For example, the thread can be used in laparoscopic interventions, particularly for fixing meshes, for example, hernia meshes, prolapse meshes or urinary incontinence meshes. Accordingly, a further aspect concerns the use of the thread for producing a fixing means for implants, in particular textile implants, preferably meshes. A further possible area of use of the thread is in the formation of anastomoses, in particular vascular or intestinal anastomoses. A further area of use of the thread concerns the production of fixing means for implants, in particular textile implants, preferably meshes. For further features and details, reference is made to the preceding description.

A thread, in particular a surgical thread, is made available which is suitable especially for use as a knotless or self-fixing surgical suture material. The main body of the thread has a coating that can be removed by liquids, preferably body fluids, and that preferably covers, in particular embeds, all of the anchoring structures formed on the surface of the main body of the thread. The outer surface of the thread is preferably made smooth as a result of the coating. In this way, the thread can be pulled gently and with the least possible trauma into a biological tissue because the anchoring structures do not come into contact with the tissue during this procedure. It is therefore not necessary to use insertion devices as described in the introduction. This allows a physician or surgeon to maneuver the thread in a manner largely free of complications and risks.

Moreover, by virtue of the coating, a physician or surgeon also has more time to position the thread correctly from the medical or surgical point of view. If appropriate, the thread can also be repositioned several times, without this resulting in additional tissue trauma. It is particularly advantageous if the anchoring structures are designed elastically or have a certain elasticity such that they right themselves preferably independently after removal of the coating.

Further features will become clear from the following description of preferred examples and to the descriptions of the drawings. Individual features can be realized either singly or severally in combination.

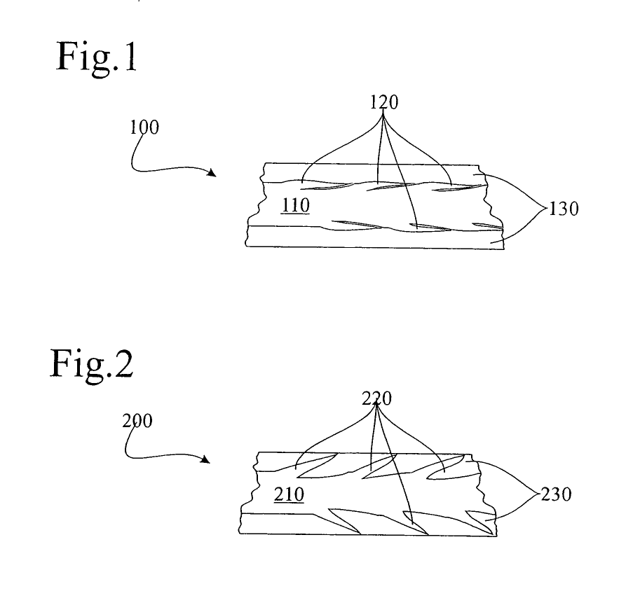

The thread 100 shown schematically in FIG. 1 has a main body 110 and anchoring structures 120 formed on the surface of the main body 110 of the thread. The main body 110 of the thread is preferably covered completely by a coating 130. The anchoring structures 120 are fixed by the coating 130 in a position in which they bear as tightly as possible on the surface of the main body of the thread. The anchoring structures 120 can be formed, for example, as cuts on the surface of the main body 110 of the thread. The coating 130 has the purpose of ensuring that the thread can be pulled into a biological tissue with the least possible trauma and can be pulled through the biological tissue with the least possible trauma. The coating 130 can preferably dissolve in or upon contact with body fluids. Ideally, however, the coating material dissolves only after a delay such that the thread 100 can, if necessary, be repositioned by a physician or surgeon and can, if appropriate, also be repositioned several times. Alternatively or in combination, the coating 130 can also be removed by flushing liquids suitable for this purpose. Suitable flushing liquids are in principle all aqueous biocompatible liquids. Examples that may be mentioned are physiological buffer solutions, electrolyte solutions, salt solutions or sugar solutions. The flushing liquids can, if appropriate, contain active substances, for example, antimicrobial, disinfecting and/or anti-inflammatory active substances.

The thread 200 shown schematically in FIG. 2 likewise has a main body 210, and anchoring structures 220 on the surface of the main body 210 of the thread. The anchoring structures 220 are covered by a coating 230 of the main body 210 of the thread such that the anchoring structures 220 are embedded in the coating 230 and are fixed by the coating 230 in a position protruding from the surface of the main body of the thread. For further features and details, reference is made to the description of FIG. 1.

### EXAMPLES

### 1.1 Production of a Barbed Thread Made of Poly-Para-Dioxanone

An undrawn monofile spinning thread made of poly-para-dioxanone was produced by thermoplastic extrusion. The monofile thread had a diameter of 0.95 mm Afterwards, 50 cuts were made into the undrawn spinning thread by means of a cutting apparatus. The cuts were made under a cutting angle of 20°, in a cutting depth of 31% in relation to the thread's diameter and in a distance to each other of 0.28 mm. The monofile thread was rotated around its longitudinal axis under 120° after each cut. Thus, a helix-shaped arrangement of cuts was formed on the thread. The cut-in thread was drawn to the fourfold of its original length in a slit heater at a temperature of 70° C. Thus, protruding barbs were formed. The self-anchoring monofile thread had a linear tensile strength of 35.8 N at an elongation of break of 29% and a diameter of 0.49 mm in thread areas where no barbs were present. The heads of the protruding barbs protruded about 0.27 mm from the main thread body.

The thread was attached at one end to a G21-cannula (without luer-lock) to investigate the pull-out strength in relation to animal tissue. The thread was attached to the G21-cannula such that the barbs were orientated away from the needle. Thus, it was ensured that the barbs did not block when the thread was pulled into the animal tissue (swine abdomen with a callosity, thickness about 2 cm). The pull-through strength in the slip direction amounted to 1.1 N. After pulling the thread into the tissue, a pull-out strength of 8.4 N was measured by means of a tensile testing machine.

### 1.2 Production of a Tube Made of Polyvinylalcohol (PVA)

A tube was extruded from a thermoplastic processable PVA (type Mowiflex TC, company Kuraray). The extrusion was performed by a Haake twin-screw extruder (TW100), a catheter nozzle (ring diameter 3 mm, support air nozzle 1.0 mm) without spinning pump using a screw rotational frequency of 10 U/min and a haul-off speed of 5 m/min under support air (nitrogen, 15 scale parts) at a nozzle temperature of 188° C. The extruded tube was spooled on a drum winder in a single-layer. The tube had a lumen diameter of 1.01 mm and a wall thickness of 0.18 mm.

### 1.3 Drawing of the Tube Produced According to Example 1.2 on the Drawn Barbed Thread as Produced According to Example 1.1

The undrawn PVA-tube according to Example 1.2 was cut to a length of 15 cm. The thread was pulled into the cut tube, wherein thread areas having no barbs were pulled into the tube at first. Further, the thread was pulled into the cut tube such that the barbs were orientated in the opposing direction. Afterwards, the thread's portion having barbs were placed within the tube such that the portion protruded into the lumen of the tube over a length of 5 cm starting from one tube end. At this end, the tube together with the thread was fixed in a clamp. Subsequently, the tube (nearly the tube) having the barbed thread within its lumen was drawn at maximum in a heating channel at a temperature of 80° C. Due to the reduction of the lumen diameter and the wall thickness, a sheath having a thickness of 0.09 mm was formed on the barbed thread. Thus, the barbs were pressed on the surface of the thread and at the same time encased by the sheath. The tube could not be mechanically separated from the thread. The surface of the tube-thread-construct was slightly corrugated. In this situation, the barbs did not show any anchoring function.

Afterwards, the PVA-sheath was cut over a length of 2 mm away from the thread body to attach the thread body to a G21-cannula.

When pulling through a swine abdomen with callosity, it turned out that the pull-through strength in slip direction was reduced to 0.3 N, due to the sheath. Further, a pull-out strength in blocking direction of the barbs amounted to 0.7 N. This advantageously offers the possibility to reposition the thread within the tissue, if appropriate, as long as the sheath is intact. The experiment was repeated after the meat was stored in an aqueous medium for a time of 5 min. The corresponding pull-out strength amounted to 5.6 N. After further 5 min, the pull-out strength amounted to 6.9 N which was close to the pull-out strength of the thread without sheath, which means that the sheath was basically dissolved.

### 1.4 Common Drawing of the Cut-in Thread According to Example 1.1 and the PVA Tube According to Example 1.2

The cut-in, yet undrawn thread according to Example 1.1 was pulled into the undrawn tube according to Example 1.2. After cutting to a length of 10 cm, the tube and thread commonly were drawn to maximal length in a heating channel at a temperature of 80° C. The result was basically the same as obtained in Example 1.3. Further, the pull-through strength and pull-out strength were basically identical to the corresponding results of Example 1.3. Furthermore, when immersing the tube-thread-construct in water at a temperature of 22° C., it turned out that the tube was basically dissolved after 4 min.

1. Thread for use as a knotless or self-fixing surgical suture material comprising;
a main body; and
anchoring structures formed on a surface of the main body of the thread and are used for anchoring in human or animal tissues,
wherein at least some of the anchoring structures are at least partially covered by a coating of the main body of the thread, which coating is removable by liquids.

2. The thread according to claim 1, wherein at least some of the anchoring structures are completely embedded in the coating.

3. The thread according to claim 1, wherein at least some of the anchoring structures are fixed by the coating in a position protruding from the surface of the main body of the thread.

4. The thread according to claim 1, wherein at least some of the anchoring structures are fixed by the coating in a position bearing on the surface of the main body of the thread.

5. The thread according to claim 1, wherein the coating can dissolve in liquids.

6. The thread according to claim 1, wherein the coating is formed as a sheath on the surface of the main body of the thread which completely surrounds the main body of the thread.

7. The thread according to claim 1, wherein the coating has a radius component of 5 to 100% relative to a radius of the main body of the thread.

8. The thread according to claim 1, wherein the coating is a film, sleeve or membrane.

9. The thread according to claim 1, wherein the coating causes the thread to have a friction-reducing substantially flat outer surface.

10. The thread according to claim 1, wherein the coating has a porosity of 30 and 90%, relative to the total volume of the coating.

11. The thread according to claim 1, wherein the anchoring structures are formed as cuts in the surface of the main body of the thread.

12. The thread according to claim, wherein the anchoring structures are barb-shaped, escutcheon-shaped, shield-shaped, scale-shaped, wedge-shaped, thorn-shaped, arrow-shaped, V-shaped and/or W-shaped, on the surface of the main body of the thread.

13. The thread according to claim 1, wherein the anchoring structures are formed unidirectionally on the surface of the main body of the thread.

14. The thread according to claim 1, wherein the anchoring structures are formed bidirectionally on the surface of the main body of the thread, in which case the anchoring structures for a first thread portion are formed in a direction of another, second thread portion, and anchoring structures for the other, second thread portion are formed in a direction of the first thread portion.

15. The thread according to claim 1, wherein anchoring structures on the surface of the main body of the thread have a periodically changing orientation.

16. The thread according to claim 1, wherein the coating softens upon contact with body fluids and preferably forms a low-viscosity melt.

17. The thread according to claim 1, wherein the coating comprises a biocompatible oligomer and/or polymer selected from the group consisting of proteins, polysaccharides, polyhydroxyalkanoates, oligohydroxyalkanoates, derivatives thereof, and combinations thereof.

18. The thread according to claim 17, wherein the material is selected from the group consisting of polyvinyl alcohol (PVA), polyvinylpyrrolidone, collagen, gelatin, elastin, albumin, dextran, amylose, amylose pectin, starch, chitosan, carboxymethyl cellulose, hydroxyethyl cellulose, hyaluronic acid, chondroitin sulphate, derivatives thereof, and combinations thereof.

19. The thread according to claim 17, wherein the polyhydroxyalkanoates are selected from the group consisting of polylactide, polyglycolide, polytrimethylene carbonate, polycaprolactone, poly-p-dioxanone, copolymers thereof, and mixtures thereof.

20. The thread according to claim 17, wherein the oligohydroxyalkanoates are at least one selected from the group consisting of lactide, glycolide, trimethylene carbonate, caprolactone and p-dioxanone.

21. A knotless or self-fixing surgical suture material comprising the thread according to claim 1.

22. The thread according to claim 1, further comprising on at least at one end a surgical insertion instrument.

23. The thread according to claim 1, which is a mass thread.

24. The thread according to claim 1, which is a tube.

25. A surgical implant comprising at least one thread according to claim 1.

26. A surgical kit comprising at least one surgical insertion instrument and at least one thread according to claim 1.

27. A method for producing a thread according to claim 1, comprising applying a coating that is removable by liquids to a surface of a main body of the thread, on the surface of which main body anchoring structures are formed to anchor in human or animal tissues.

28. The method according to claim 27, wherein the coating is applied to the surface of the main body of the thread by sheathing extrusion.

29. The method according to claim 27, wherein the coating is applied to the surface of the main body of the thread by the following steps:
a) immersing the main body of the thread in a solution or suspension that contains a coating material;
b) removing the main body of the thread from the solution or suspension; and
c) drying the thread body to form the thread.

30. (canceled)

31. A mesh comprising a plurality of threads according to claim 1.
